(19) United States
(12) Patent Application Publication
(10) Pub. No.: US 2005/0203476 A1
(43) Pub. Date: Sep. 15, 2005

(54) Wipe 'n diaper

(76) Inventor: Nicky Sanchezis Stegall, Marietta, GA (US)

Correspondence Address: Nicky Stegall, Route 1, P.O. Box 139C, McRae, GA, 31055, US

(21) Appl. No.: 10/800,338

(22) Filed: Mar. 15, 2004

(51) Int. Cl. A61F 13/15; A61F 13/20

(52) U.S. Cl. 604/385.06

(57) ABSTRACT

A disposable diaper, including a prepackaged, inner bounded pouch, containing items used generally when changing a diaper, such as baby wipes and a variation of baby powder and optional medicinal cream, such as diaper rash ointment. The affixed items are found in the pouch and are accessible to the user by means of a zipper style closure keeping the wipes from drying out.

### CROSS-REFERENCES TO RELATED APPLLICATIONS

Prior art known dealing with both disposable diapers and combination packages include
U.S. Pat. No. 4,738,678 granted to Paulis on Apr. 19, 1988, which teaches the concept of a disposable diaper and retention of a wipe to clean the user (individual administering the child care) after each diaper use.
U.S. patent 2002/0156448 granted to Stegar et al. on Oct. 24, 2002, which teaches the concept of a sanitary napkin with the addition of a wet wipe, used to clean the user after each use.
U.S. Pat. Des. No. 334,426 granted to Meis on Mar. 30, 1993, which teaches the concept of a disposable, diaper with a combined diaper and wipe attachment.
U.S. Pat. No. 5,304,158 granted to Webb on Apr. 19, 1994, which teaches the concept of a disposable diaper and an included changing pad.
U.S. Pat. No. 4,964,859 granted to Feldman on Oct. 23, 1990, which teaches the concept of a disposable diaper, changing pad, and disposable container combination.

### BACKGROUND OF THE INVENTION

Disposable diapers have proven to be a valuable asset to infant care. It is a fact, however, that there are more products needed other than diapers during the diaper changing process. These materials may include, but are not limited to, baby wipes, baby powder, or even diaper rash ointment. With more insight into the invention, it was further discovered that the inclusion of these materials into the diaper would dramatically decrease the bulk of a baby's bag. The practicality of this product is evident with the addition of these products to the diaper allowing for immediate and easy accessibility when needed.

### BRIEF SUMMARY OF THE INVENTION

A pouch containing infant care wipes is affixed to the diaper making the changing of diapers an easier process. The contents in the pouch are accessible by a zipper style closure. As a variation, a small single use of baby powder and/or medicinal ointment could be included in the pocket. The pocket containing these items would be enclosed in such a manner so the wipes would not dry out, and the other materials wouldn't be exposed to the wipes. This product solves the problem of having to pack both diapers and wipes, or even ointment, which are all space-consuming products.

### BRIEF DESCRIPTION OF THE SEVERAL VIEWS OF THE DRAWING

FIG. 1 is a bottom plan view, shown sideways, of the diaper.

FIG. 2 is an enlarged view of the pouch.

FIG. 3 is a side view taken of the closed diaper.

FIG. 4 is a side view of an opened diaper.

### DETAILED DESCRIPTION OF THE INVENTION

If manufactured, this product would be produced similarly to that of conventional diapers, including all of the modern features. The features included would be a gathered waistband, leg gathers, stay-dry liners, highly absorbent materials, adhesive tabs, and even the decorative outer linings of the diaper. A plastic pocket containing any suitable number of wipes would be secured in the diaper. The pouch, on the sides and bottom would be impermeable, allowing for the pre-moistened wipes to stay moist, as well as preventing any moisture from reaching any other sections of the diaper. This pocket's zipper-style closure would allow for easy access.

This invention is appealing because all of the materials needed to change a diaper would be easily accessible, and it wouldn't be necessary to add diapers, a box of wipes, a bottle of baby powder, and even a tube of ointment to the bag, rather all of these materials will be at the care provider's disposal.

### DETAILED DESCRIPTION OF SEVERAL VIEWS OF THE DRAWINGS

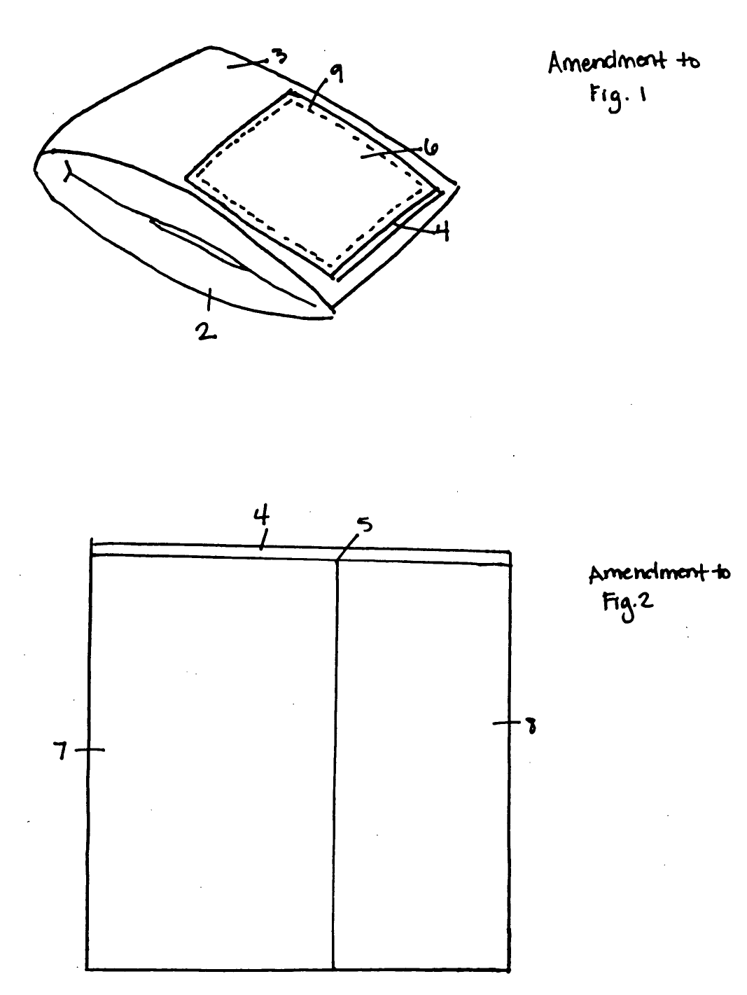

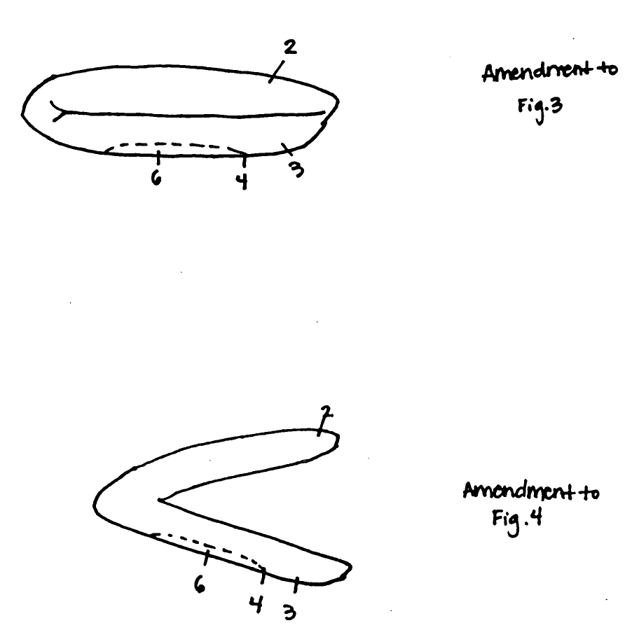

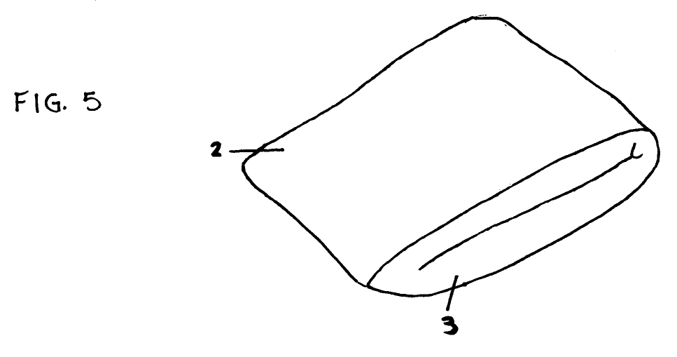

As seen in FIG. 1 the disposable diaper is shown in its folded condition, with the bottom 3 facing up and the top 2 facing down. Affixed to the diaper is a pouch 6 containing the premoistened wipes. The pouch is sealed by a zipper style closure 4 allowing for easy access to the wipes. The outer material 9 encompassing the entirety of the diaper hides the pouch keeping it from plain sight. Demonstrated in FIG. 2 are the contents of the pouch in its varied form. The separation 5 depicts the hermeneutically sealed sectioning of the pouch. The larger portion 7 showing the side with the sides and the smaller 8 showing positioning of optional powder or medicinal cream. FIGS. 3 and 4 further show the pouch 6 in relation to the diaper. As shown in the figures the pouch 6 is located within the diaper with the outer material 9 covering it.

1. A disposable diaper and wipe combination including premoistened baby wipes containing:
said premoistened wipes included inside the affixed sealant packet not allowing the wipes to dry out or come into contact with other sections of the diaper,
the pocket added in a manner that in located inside the diaper whereas not to be seen when looking at the back of the diaper,
the extension of this pocket in a size allowing for at least five wipes, folded in on themselves, to be included,
means of accessing the wipes by opening a zipper style closing; this is the same means in closing the pouch.

2. An optional addition of other materials needed in the baby changing process (i.e. medical ointment or baby powder) affixed in the same manner as the wipes.

3. A variation allowing for addition of this pouch in a manner that may be removable (location at the manufacturer's discretion) allowing its purpose to be served at any time be it during of after the changing process.
